(19) United States
(12) Patent Application Publication
(10) Pub. No.: US 2003/0027203 A1
(43) Pub. Date: Feb. 6, 2003

(54) Biomolecular processor

(76) Inventor: Robert E. Fields, Palo Alto, CA (US)

Correspondence Address: FISH & NEAVE, 1251 AVENUE OF THE AMERICAS, 50TH FLOOR, NEW YORK, NY, 10020-1105, US

(21) Appl. No.: 10/243,521

(22) Filed: Sep. 12, 2002

Related U.S. Application Data
(63) Continuation of application No. 09/381,603, filed on Sep. 22, 1999, now abandoned.
Application No. 09/381,603 is a 371 of international application No. PCT/US98/06029, filed on Mar. 23, 1998.
(60) Provisional application No. 60/041,237, filed on Mar. 24, 1997.

(51) Int. Cl. C12Q 1/68; C07H 21/02; C07H 21/04; C12N 1/08; C12M 1/34

(52) U.S. Cl. 435/6; 435/270; 536/25.4; 435/287.2

(57) ABSTRACT

A process and apparatus for isolating and purifying nucleic acids and other target molecules directly from blood, plasma, urine, cell cultures and the like by totally automated means, without centrifugation, aspiration or vacuum. After mixing a sample containing target molecules with a test reagent in an environmentally isolated compartment, target molecules are adsorbed onto a binding material and eluted in a small volume using an elution reagent. A preferred embodiment purifies nucleic acids and automatically detects target sequences from a sample of fresh blood.

This is a 371 of PCT/US98/06029, filed Mar. 23, 1998, which claims priority to Provisional Application Ser. No. 06/041,237, filed Mar. 24, 1997, now abandoned.

### FIELD OF THE INVENTION

The invention relates to laboratory and clinical instruments, procedures for isolation and purification of target molecules from biological fluids, and apparatus for combining target molecules with other components of an assay mixture.

### BACKGROUND OF THE INVENTION

In the United States more than 500 thousand people per year die from sepsis, an often-fatal complication following surgery, which requires identification of a bacterial strain before effective treatment can be given. Identification by cell culture can take up to two weeks. A series of rapid nucleic acid-based diagnostic tests, if available, could be used to determine, first the family, then the strain of a pathogen. Such tests are not in routine use, however, owing to the present labor intensive requirements for isolating nucleic acids and the absence of high speed nucleic acid (NA) tests.

The emergence of drug resistant strains of viruses and bacteria requires that strains be identified before effective medication can be prescribed. The culture and identification of strains of Mycobacteria, for example, is often difficult and prolonged, during which time a patient, if untreated, may infect others. A rapid NA-based test for M. tuberculosis with differentiation of strain could significantly assist in the worldwide control of tuberculosis.

Latent cancerous tissues can be detected from aberrant cellular gene sequences and effective treatment can be administered on the basis of knowledge of such sequences.

NA tests that provided rapid information on viral genotypes in patient blood would assist the effective management of HIV infection by allowing antiviral drug combinations to be changed in response to the appearance of mutant genotypes of HIV.

The present invention automates the purification of NA from blood or plasma and provides for rapid combination of purified NA with other components of assay mixtures for detecting or determining the quantity of particular NA sequences using techniques such as the polymerase chain reaction (PCR), the Ligase Chain Reaction (LCR) or using gene chip technology. Target molecules other than nucleic acids can be purified and identified by the invention, and the invention is in no way restricted to use with nucleic acids.

All purification and detection procedures take place in sealed compartments provided by the invention. This feature makes the invention particularly advantageous for use as a clinical diagnostic tool to provide rapid patient information with a minimal risk of contamination to healthcare workers and minimal risk of test contamination by “carryover” DNA.

Current Nucleic Acid Purification Techniques

Nucleic acids have been isolated and purified using a wide variety of reagents and techniques, depending on the source of the NA and the use that is to be made of purified NA. Procedures for isolating NA from biological samples include 1) cell lysis using a combination of mechanical disruption, detergents, proteolytic enzymes, chaotropic agents or other chemicals, followed either by 2) extraction of the lysate using phenol or another organic solvent, or 3) centrifugation to clear the lysate, followed by 4) precipitation of the NA, or 5) adsorption of the NA onto solid phase materials, followed by 6) washing the solid phase materials, and 7) eluting bound NA from solid phase, or 8) centrifuging the precipitated NA and 9) resuspending the pellet, or 10) purifying the NA by gel electrophoresis, 11) cutting the band of NA from the gel and 12) recovering the NA from the gel or 13) obtaining purified NA using two or more rounds of cesium chloride density gradient centrifugation, with 14) recovery of NA, and 15) removing salts or changing the NA buffer using dialysis, gel chromatography or spin columns.

These procedures are carried out manually by trained personnel using vortexers, hand actuated pipettors, centrifuges, gel electrophoresis apparatus, hot water baths, vacuum aspiration for the removal of supernatants after centrifugation etc. The reproducibility of a lab's results often depends on the liquid handling skills of a particular technician, and the variable delays that may occur between multiple steps when a technician is carrying out several procedures in the laboratory at one time.

To prevent enzymatic destruction of NA molecules during purification, enzyme inhibitors have been used to block RNAse and DNAse digestion in the lysates. Cox (in Cox, R. A. (1968) “The use of guanidinium chloride in the isolation of nucleic acids” Methods Enzymol. 12B: 120.) showed that a high molar concentration of guanidinium salt not only disrupts secondary cellular structures, facilitating the liberation of nucleic acids, but it also inhibits the action of RNases owing to its action as a powerful chaotrope. As a result, guanidinium salts, particularly guanidinium thiocyanate, have become established in wide use in NA purification procedures.

Boom (in Boom, R., Sol, C. J. A., Salimans, M. M. M., Jansen, C. L., Wertheim-van Dillen, P. M. E., and van der Noordaa, J. (1990). “Rapid and simple method for purification of nucleic acids” J. Clin. Microbiol. 28 (3), 495.) studied the feasibility of combining a starting sample with a suspension of silica particles or diatoms in guanidinium thiocyanate (GuSCN). The recovery of NA from starting materials using this method was found to be problematic. Boom's U.S. Pat. No. 5,234,809, assigned to Akzo, N. V., teaches the mixing of a NA containing sample with a chaotropic substance and a nucleic acid binding solid phase, separating the solid phase, and washing the solid phase. The NA purification process as taught by Boom is carried out using manual procedures such as vortexing a suspension of silica particles before manually adding starting material, centrifuging, removing supernatants by vacuum aspiration, pipetting wash solutions, vortexing pellets to resuspend them in wash solution before further centrifugation, incubating a sample tube in a water bath or heating block, etc.

Centrifugation based procedures can be simplified by the use of spin-tubes or spin columns, in which target molecules, after liberation using detergents, enzymes or chaotropes, are bound to a solid phase material at the bottom of a tube which is placed in a second centrifuge tube. The solid phase is washed by applying a wash solution on top and centrifuging the assembled pair of tubes, which causes the wash solution to pass through the solid phase and to be collected in the second centrifuge tube. Target molecules are collected in a centrifuge tube by applying an elution solution to the top of the solid phase, and centrifuging the pair of tubes to collect the eluate.

Although the aspiration step is eliminated using spin-tube techniques, the spin-tube method is still labor intensive and unsuitable for automation. As a result of the relatively long time that is required to complete these and other conventional NA purification protocols and the labor intensive nature of the protocols, these procedures add to the total cost of carrying out a NA diagnostic tests.

Attempts have been made to automate parts of nucleic acid purification techniques such as dispensing reagents, diluting, aspiration and mixing of liquids using digital syringe pumps, pipettors and robotics. U.S. Pat. Nos. 4,488,241 and 4,510,684 assigned to Zymark Corp. and U.S. Pat. No. 5,104,621 assigned to Beckman Instruments, Inc. disclose robotic systems having interchangeable tools and hands for manipulating cooperating embodiments of laboratory devices, permitting otherwise manual procedures to be performed by the robotic apparatus, according to a computer program that is entered by the user. These patents teach the use of general purpose robotic systems intended for use for a wide variety of chemical and laboratory procedures. These inventions have been designed to be as flexible as possible, and do not specifically provide for rapid isolation of nucleic acids or other target molecules or for combining purified NA with other components of an assay mixture.

The isolation of nucleic acids from liquids that contain infectious agents such as bacteria and viruses requires stringent sample handling techniques to avoid contamination and infection of health care workers. To minimise such risks NA purification techniques involving open pipetting and centrifugation specify that such procedures are carried out in a containment hood.

The requirement for such special containment facilities and other instruments needed for NA purification presently restrict these procedures to laboratory and clinical sites. Blood, for example, collected in remote areas of the world, must be rapidly transported to urban areas for NA purification and testing, since the blood contains enzymes that degrade the NAs that are to be isolated. If NAs could be isolated from blood in the field, the resulting purified NAs would be chemically stable and could be transported back with a reduced risk of degradation.

It would therefore be desirable to provide a biomolecular processor that did not require the use of manual pipetting, centrifugation and that was less labor intensive than current methods of purification of nucleic acids.

It would also be desirable to provide a biomolecular processor that secluded the nucleic acid containing sample from the environment while purification took place, so as to reduce the risk of infection of workers and contamination of the environment.

It would also be desirable to provide a biomolecular processor that is capable of isolating nucleic acids or other target molecules from fresh whole blood, plasma, sputum, urine, semen, tissue samples, feces, bacterial or cell cultures.

It would also be desirable to provide a biomolecular processor that is capable of combining purified nucleic acids or other target molecules with components of a test for detection or quantification of such target molecules.

It would also be desirable to provide a biomolecular processor that is capable of processing single samples or a multiplicity of samples and that is completely automated in its operation.

### OBJECTS OF THE INVENTION

A principal object of the present invention is to overcome the limitations of previous methods for isolating NA by combining a nucleic acid containing sample with a lysis reagent in an incubation vessel from which all nucleic acid binding materials are excluded, in order that nucleic acids may be completely liberated into the lysate solution before recovering the NA.

A further object of the invention is to eliminate all requirements for manual or skilled techniques such as vortexing, pipetting, aspiration, centrifugation, the use of spin-columns, extraction by organic solvents, suspension of pellets, or the use of vacuum manifolds for the purification of NA.

A further object is to provide means for automatically withdrawing a sample into the invention through a port that may be closed immediately afterwards to prevent contamination of the environment by the sample.

A further object of the invention is to provide means for thorough mixing of the sample with a lysis reagent within the invention; means for heating and controlling the temperature of said lysate mixture; means for removing said lysate from the incubation chamber; means for causing said lysate to pass through a solid phase matrix upon whose surface target molecules may be bound and thereby removed from said lysate; means for washing said solid phase matrix using suitable wash liquids; means for heating and controlling the temperature of said wash solutions; and finally, means for scrubbing said solid phase matrix and eluting target molecules using a suitable elution liquid; with means for heating and controlling the temperature of said elution liquid.

It is a further object of the invention to provide for isolation and purification of nucleic acids or other target molecules directly from blood, plasma, sputum, urine, semen, tissue samples, feces, bacterial or cell cultures by a fully automated means.

It is a further object of the present invention to provide a means for the rapid combination of purified NA or other target molecules with other components of a NA or other assay for detection or quantitation of said target molecules.

It is yet another object of the invention to provide a means for isolation, purification and detection of NA or other target molecules that is rapid and does not require the use of any laboratory apparatus other than the invention itself.

### SUMMARY OF THE INVENTION

In one aspect, the invention provides a device and an automated method for isolation and purification of nucleic acids or other target molecules from raw starting materials. A preferred embodiment for isolation and purification of total NA provides an apparatus into which starting material, such as whole blood, plasma, or a suspension of cells is drawn, and out of which purified nucleic acids are automatically dispensed, without need for pipetting, centrifugation or manual labor.

The invention may process a single sample at a time or it may process a multiplicity of samples using a dispensing apparatus cooperating with an array of tubes or wells in a microtiter plate, with means for heating and mixing contents of the said tubes or wells, in which samples containing nucleic acids or other target molecules are combined with lysis reagents, and from which purified target molecules may be obtained, with no requirement for manual pipetting, centrifugation, aspiration or manual labor.

In another aspect, the invention may combine a portion of purified nucleic acids with components of an assay, deliver the said mixture of components into a thermal controller, a gene chip or detection device to enable target molecules to be identified or the quantity of said molecules to be determined. Such target molecules for isolation and purification may be of a single type, a combination of RNA, DNA, proteins or of other molecules.

In yet another aspect, the invention provides test kits consisting of disposable component devices and reagents that cooperate with the mechanical portion of the invention to enable target molecules to be purified, detected or quantitated, either from a single test sample or from a multitude of test samples, in such manner as to minimize the risk of contamination of the environment, or of the mechanical portion of the invention.

### BRIEF DESCRIPTION OF THE DRAWINGS

The foregoing and other objects and advantages of the invention will appear from the description which follows. In the description reference is made to preferred embodiments of the invention. Such embodiments do not necessarily represent the full scope of the invention.

FIG. 1 is a perspective view of the invention.

FIG. 2 is a plan view of a disposable extractor device of the invention preferred for use for processing individual samples of target molecules.

FIG. 3 is a plan view of valve components for the invention.

FIG. 4 is a plan view of a disposable extractor device of the invention preferred for use for single sample processing of target molecules, wherein the sample is automatically withdrawn from a capillary tube.

FIG. 5 is a schematic diagram of the operational hardware of a preferred embodiment of the invention.

FIG. 6 is a plan view of a disposable device of the invention preferred for use for single sample processing of target molecules, wherein purified NAs are automatically combined with components of an NA assay mixture before being introduced into a PCR thermal cycler or other NA assay device.

FIG. 7 is a perspective view of the mechanical hardware component of a preferred embodiment of the invention used for processing a multiplicity of samples.

FIG. 8A is a section view of a target molecule extraction pipettor tip constructed in accordance with an embodiment of the invention preferred for use with a multiplicity of samples.

FIG. 8B is a section view of another preferred embodiment of a target molecule extraction pipettor tip.

FIG. 9A, B, and C show the extraction, wash and elution steps for an extraction pipettor tip

FIG. 10A is a perspective view of a 96-well microtiter plate formed in a plastics material, constructed in accordance with the invention.

FIG. 10B is a plan view of the array of wells shown in FIG. 10A.

FIG. 10C is a section view taken along the line 2-2′ of FIG. 10B.

### DETAILED DESCRIPTION OF THE DRAWINGS

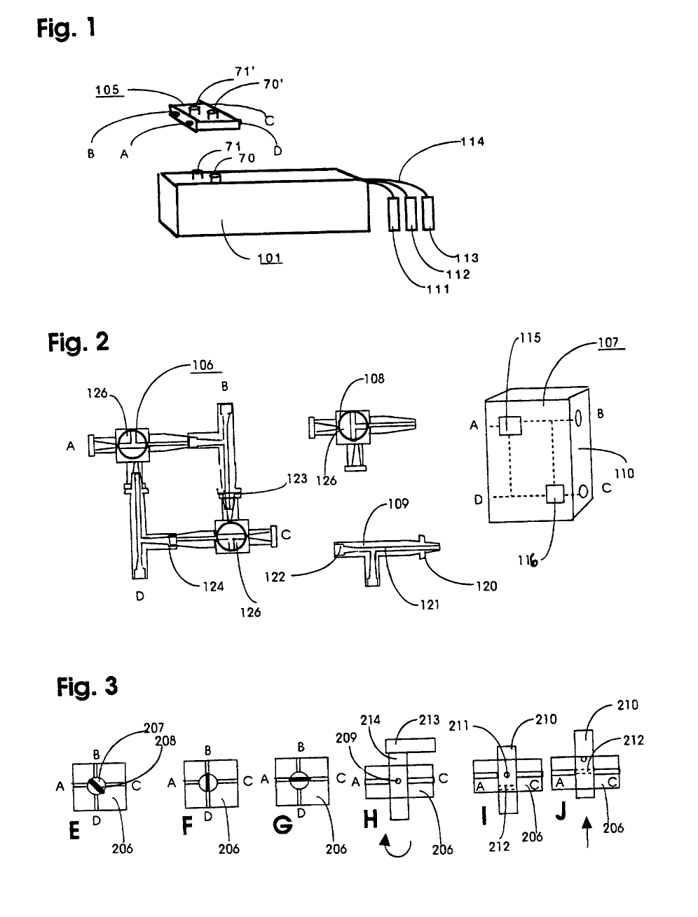

With reference to FIG. 1, a preferred embodiment of the invention comprises mechanical actuator 101 that is able to engage a single-use, disposable device 105 by means of adapters 70, 71 which engage cooperating adapters 70′, 71′ in device 105. Reagents 111, 112, 113 may be withdrawn into actuator 101 via tubes 114. Device 105 is provided with port A which may be opened to receive a sample for analysis, port B which may be connected to an incubation chamber, a thermal cycler or other treatment vessel, port C from which purified target molecules may be dispensed for collection or for further analysis, and port D through which reagents may enter from actuator 101 for the purification and identification of target molecules. Molecules contained in a sample are wholly contained within the device 105 and may not enter or contact surfaces of the actuator 101.

FIG. 2 shows preferred embodiments of an extractor device 106 and 107, their component parts consisting of stopcock 108 and tee connector 109.

With reference to FIGS. 3E-3J, cross sections of several valve types are shown. Valve body 206, having ports at A, B, C, D which may be a selectively sealed or connected by means of moveable member 207 having one or more internal pathways 208 formed within such member.

FIG. 3E shows member 207 in a position such as to prevent an internal connection between any ports A, B, C, D. FIG. 3F shows the position of member 207 which will allow connection between ports B and D; similarly FIG. 3G shows the position of member 207 which will allow connection of ports A and C; numerous means are known for selecting pairs or combinations of ports using a moveable member 207.

FIG. 3H shows a rotary member 208 having a handle 213 with a passage 209 drilled through the member 208. Ports A and C may be connected internally by rotating member 208 to align the passage 209 with said ports. Alternatively, moveable member 210 may contain drilled passages 211 and 212 which will connect ports within the body 206 by alignment using rectilinear motion, rather than rotary motion, of member 210, as shown in FIG. 3J. The disposable device 105 of the present invention may be composed of either type of valve and such valves may have any number of selectable ports.

In FIG. 2, device 106 is made up of two stopcocks 108 joined sealingly together with two tee connectors 109 forming device 106 having four ports, A, B, C, D. Various pathways connecting the ports may be selected by turning moveable stopcock members 126 so as to align or block internal passages in the device. Preferred tee connectors 109 are formed with narrow bore internal passages 121, and with male luer fittings that contain hubs 120, female luer fittings that have internal shelves 122 which control the depth to which a mating luer component may enter, as shown at 123 and 124, thereby controlling the internal volume of assembled device 106.

Device 107 may be formed from 3 component parts, consisting of body 110, and moveable members 114 and 115 which may be moved so as to connect or seal off ports A, B, C, D according to positioning of said moveable members, as shown in FIGS. 3I and 3J.

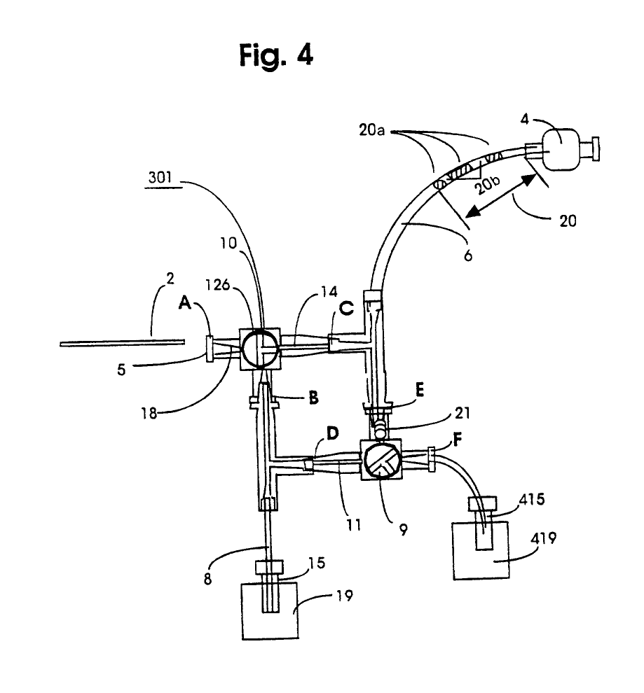

With reference to FIG. 4, a preferred embodiment of the disposable device 301 enables NA to be extracted and purified from a sample presented to the invention in a capillary tube 2. The sample makes physical contact only with disposable device 301, which is attached to mechanical actuator 101 (FIG. 1). The mechanical actuator 101 does not make contact with a sample or molecules derived from a sample. Capillary tube 2 which contains a sample containing target molecules is inserted into port 5 until the outer edges of the end of the tube are pressed into and sealed on the inner tapered surface of the port at 18. The surface is made preferably made of an elastomeric material. Valve 10 is turned so that the sample may be withdrawn from the capillary into device 301. After induction of sample from into the device, said sample is isolated from the environment by closure of valve 10. Said sample is prevented from contact with reagents in the mechanical actuator 101 by a barrier of alternating volumes of fluid 21 and air, i.e., a segmented fluid barrier, as shown at 20.

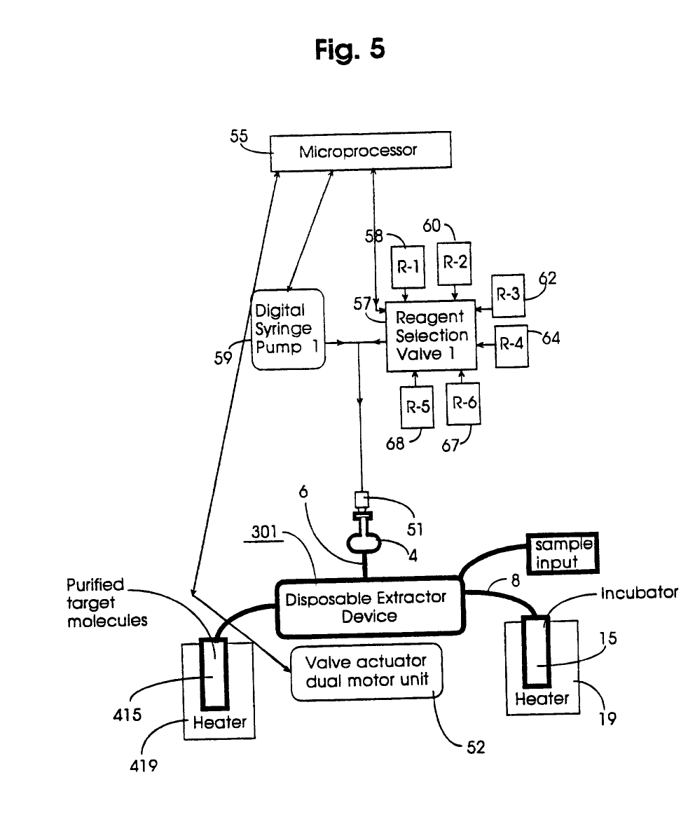

For the extraction of NAs, or other target molecules, device 301 is attached to actuator 101 via a connection between fittings 4 and 51 (FIG. 5). Microprocessor 55 causes the dual motor stopcock drive unit 52 to set valve 9 (FIG. 4)to a position in which all ports are blocked and to set valve 10 to a position in which ports B and C are connected, as shown in FIG. 4. Microprocessor 55 then causes reagent selection valve 57 to select lysing reagent, RI 58, and causes digital pump 59 to begin dispensing reagent 58 into tube 6 of disposable device 301. Continued pumping of reagent 58 will cause it to enter into tube 8 and into incubation chamber 15.

A sample collected in capillary tube 2 for extraction of NA or other target molecules is inserted into rubber seal 18. Microprocessor 55 causes the dual motor stopcock drive unit 52 to set stopcock 10 to a position in which ports A and C are connected and port B is sealed off. Microprocessor 55 then induces pump 59 to withdraw the required sample volume, which causes the contents of capillary tube 2 to be drawn into valve 10 and into passage 14. The microprocessor then acts to return valve 10 to the position in which B and C are connected and causes the contents of capillary tube 2 to be pumped through the internal passages of the device into incubation chamber 15. The digital syringe pump 59 is then able to mix the contents of 15 by rapidly withdrawing and dispensing an appropriate quantity of the mixture in a to and fro manner causing agitation mixing of the lysis reagent and sample solution in 15. According to this embodiment of the invention the incubation vial resides in a heated well of thermostatically controlled heating block 19 which heats the lysis mixture to assist in liberating nucleic acids.

It will be appreciated that any amount of lysis reagent 58 can be dispensed, followed by nitrogen gas or air, taken into syringe pump 59 from 60. Such a gas can be used to move reagents and samples through the device 301 and specific amounts of such gas can be injected in between different reagents to separate the reagents and prevent the reagents from having a common liquid interface and thereby mixing. In the summary that follows, gas is used in this manner to separate the different reagents; however in the interests of brevity, the details of valve and syringe movements necessary for the use of this gas will not be given. It will be understood that numerous combinations of different reagents and gases and of the uses of valves and syringe pumps all lie within the scope of the present invention.

When nucleic acids or other target molecules in incubation chamber 15 have been liberated, pump 59 withdraws the lysis mixture from 15 into tube 6. The internal volume of 6 has been chosen so that there is a protective space at 20 filled with a segmented fluid barrier to insulate device 50 from contact with molecules in the sample lysate which has been drawn into tube 6.

Stopcock 10 is now closed and stopcock 9 is turned so that ports E and D are connected. Pump 59 forces the lysate through target molecule adsorption filter element 21, through stopcock 9, into passage 11, and into chamber 15. Microprocessor 55 acting causes reagent selection valve 57 to select wash reagent, R3 62, and causes digital pump 63 to begin dispensing reagent 62 into device 301, through filter 21, causing said filter and bound target molecules to be washed. If needed, wash reagent R3 can be collected in flask 415 and heated by heater 419. In fact the heating of the wash reagent may be carried out before passage of lysate through the filter 21. In any case, the filter can be scrubbed using to and fro action of pump 59, and the wash liquid is collected in flask 15. Microprocessor 55 now causes reagent selection valve 57 to select elution reagent, R4 64, and to dispense R4 into device 301. When elution reagent has entered filter element 21, pump 59 facilitates the release of NA or other target molecules by alternately dispensing and withdrawing a small volume, to and fro, causing fluid turbulence within the filter element 21, before turning stopcock 9 to the position in which port E is connected to port F, and collecting the target molecules in a small volume in vial 415. Elution reagent R4 can be collected in flask 415 and heated by heater 419 before passage of lysate through the filter 21. In this case, target molecules can be eluted from the filter using a heated elution reagent.

It will be appreciated that a sample can be introduced into device 3 via a pipette tip in place of a capillary tube 2, and that a flexible tube can be provided at fitting 5 so that a sample contained in a tube may be siphoned into the device. In addition provision could be made of a protected needle device to puncture the rubber stopper of a sample tube for removal of a sample from such a sealed tube. Such variations lie within the scope of the present invention. Furthermore, disposable syringes containing reagents could be provided in place of the digital syringes and selection valves described above. Any configuration of a disposable devices, including disposable syringes, stopcocks, fittings and tubing, which contacts molecules of the sample solution during the purification of target molecules by the process described by this invention falls within the scope of the invention.

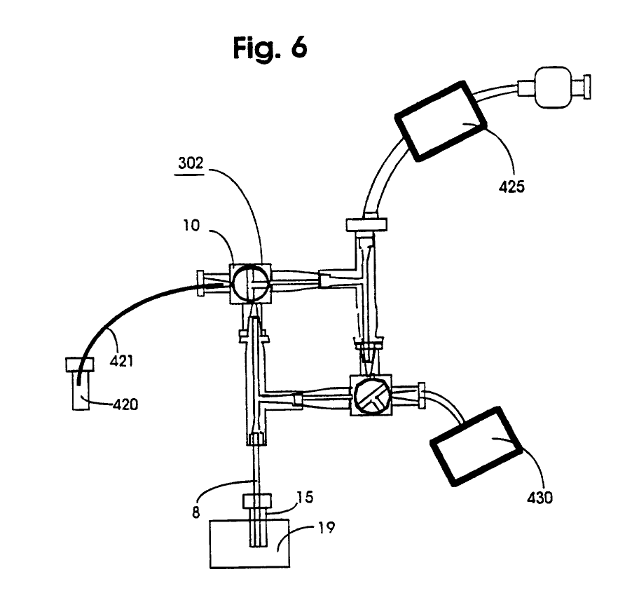

FIG. 6. is a schematic representation of a preferred embodiment of a disposable device 302 used for determination of specific nucleotide sequences in a sample or for determining the quantity of target molecules. Purified nucleic acids or other target molecules are taken up from vial 420 and combined with other components of an assay in vial 15. The volume of target molecules taken can be accurately determined using digital pump 59 in combination with valve 10. For PCR assays the target NAs are given a hot start in vial 15 by preheating the master mix solution using heater 19. After thorough mixing the assay mixture is moved into device 430, which, in the case of PCR is a thermal cycling unit for amplification of target sequences. However other sample processing devices may be used. In the case of PCR, following the amplification step, amplified products are moved into device 425, for detection of amplified products. Device 425 can be a hybridization tube or a DNA chip. Wash reagents and detection reagents can be dispensed as required, for activation of fluorescent of chemiluminescent reactions in device 425. After measurements are made, disposable device 302 is safely disposed of without contaminating the environment.

The assay and detection embodiment of the invention just described may be coupled with the target molecule purification embodiment of the invention described earlier to enable specific nucleic acid sequences to be detected and quantitated automatically from a starting sample. The coupled use of the two embodiments of the invention lies within the scope of the invention.

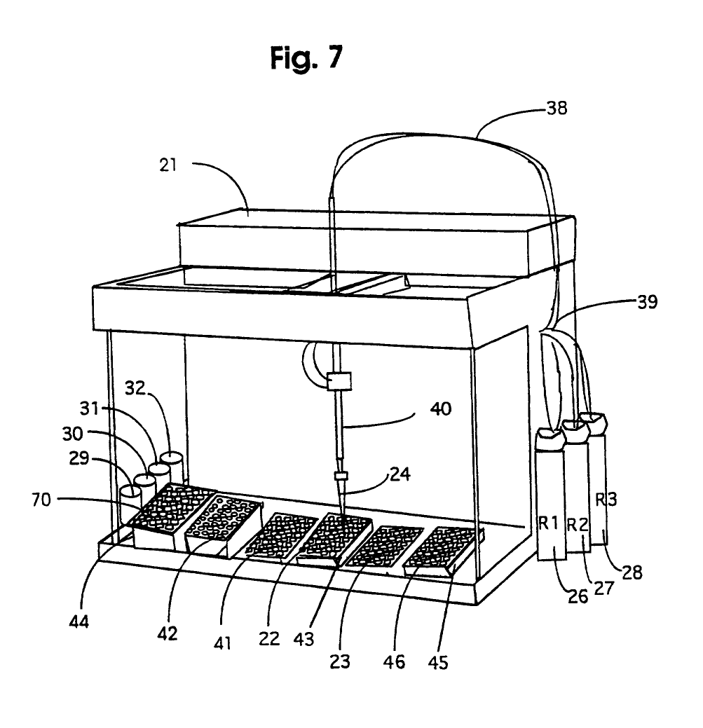

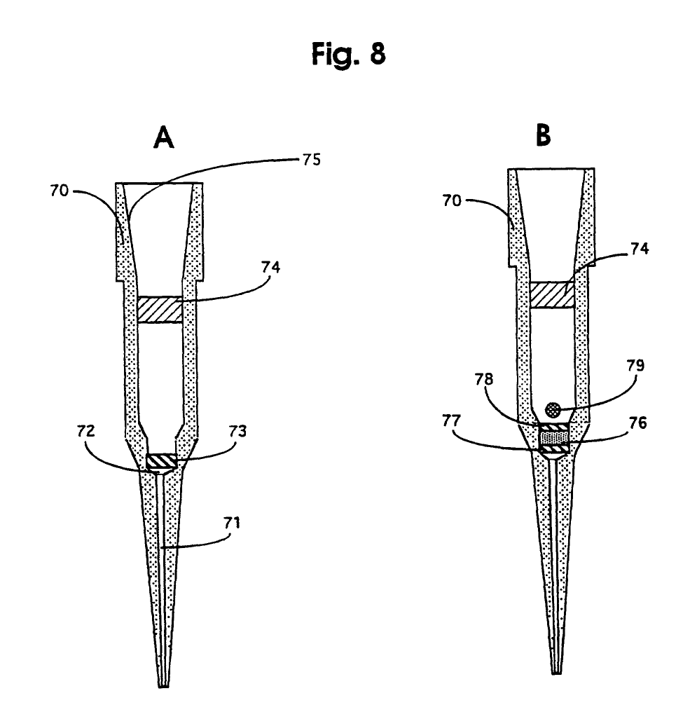

FIG. 7 shows an embodiment of the invention 21 preferred when numerous samples of blood, plasma or other nucleic acid, or other target molecule, containing material requires to be processed. Samples are presented for processing in racks or arrays of tubes 41, or in the wells of a microplate. Robotic arm 40 dispenses lysis reagent R1 26 by means of a valve and syringe pump located behind 39 passing through pipe 38 and into each incubation well in microplate 22, which sits in a heated block 43. Robotic arm 40 picks up a clean pipettor tip 24 from a storage rack of tips at 42 and transfers a portion of a first sample in 41 to an incubation well in 22. The microprocessor controller of the device mixes the sample thoroughly with the lysis reagent by turbulent aspiration of the mixture, as described previously. The pipettor tip is discarded and an a fresh tip picked up to combine a next sample with lysis reagent. After all samples have been combined and mixed with lysis reagent, and the lysates have been heated to release NA, or other target molecules, robotic arm 40 picks up a clean extractor pipettor tip 70, FIG. 8A or 8B, held in rack 44, FIG. 7.

The Extractor Tip

The body of the extractor tip 70 (FIG. 8) is preferably formed from inert plastics material, having a low internal volume entrance 71, with an internal diameter typically between 0.3 mm and 0.8 mm, a low dead volume collector cavity 72, a porous, solid phase material 73 capable of binding nucleic acids or other target molecules, and removing such molecules from solution as a lysate solution passes through porous material 73. The means of binding target molecules may be by hydrophobic, ionic or any other interaction. Preferably extractor tips 70 are also fitted with a porous hydrophobic or scavenger containing element 74, through which air may pass, but which element will not allow nucleic acids or other target molecules to pass, thereby preventing contamination of the pipettor instrument. Extractor pipettor tip 70 has a tapered fitting portion 75 to allow it to be sealingly engaged with a pipettor. FIG. 8B shows another embodiment of the invention having solid phase binding material 76, which may be in particulate form, held between inert porous supports 77 and 78. Above porous support 78 is shown reagent 79 in solid form which may contact a lysate or other liquid after it has passed through the binding material. Reagent 79 may interact with a liquid on contact, dissolving and releasing active agents such as buffering compounds or enzymes or components of assays, or it may disburse insoluble elements such as silica or derivatised latex particles into the liquid. Any type or combination of filtration or selective binding element and any type of separate reagent or particle kept in the above position as shown is within the scope of this invention.

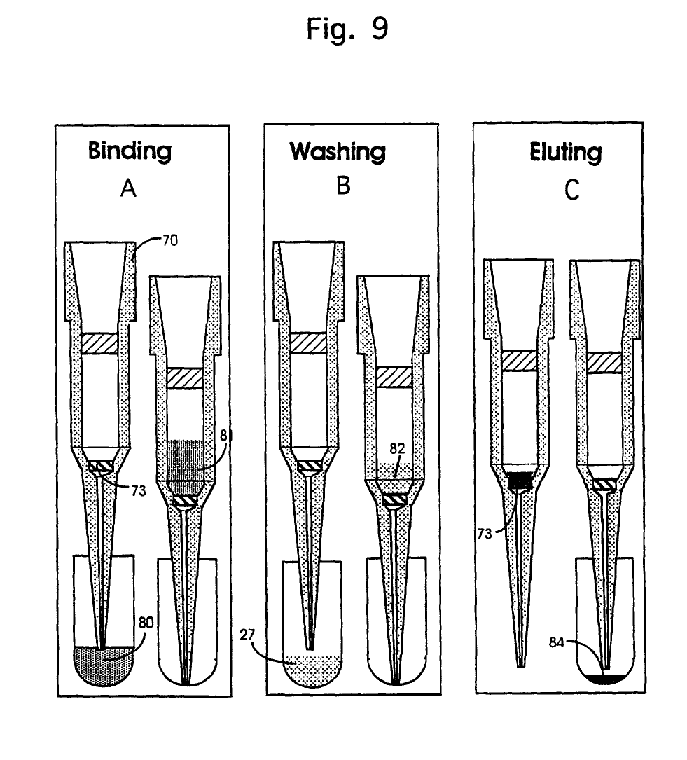

The robotic arm 40 of the invention picks up a clean extractor pipettor tip 70, held in rack 44, FIG. 7. and withdraws lysate from an incubation well in 43. The lysate may be dispensed and taken up several times to assure complete adsorption of target molecules to element 73, FIG. 9. Afterwards the lysate solution is returned to well 22 and robotic arm 40 washes the filter element 73 using wash solution R2 27 that has been previously dispensed into wells at 23. Filter element 73 may be washed multiple times, at 82, using reagents held in multiple wells at 23. Finally a small amount of elution reagent R3 28, typically 20 to 100 microliters, in one quadrant of a multiple well at 23 is used to scrub the filter 73 to and fro, to remove target molecules before dispensing the target molecules 84 into reagent tubes or assay mixtures at 44, depending on the protocol being followed.

Automated Make-Up and Storage of Assay Solutions

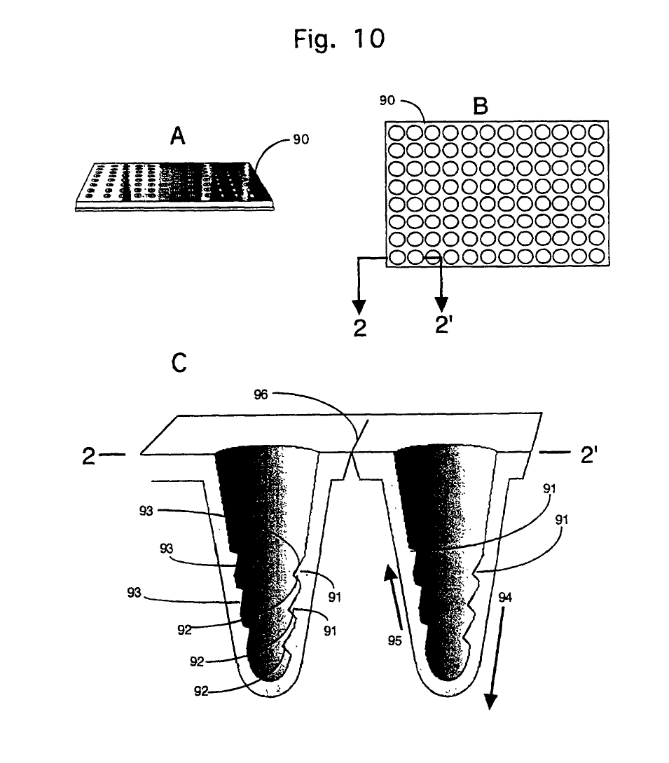

The invention comprises special tubes, strips of tubes or wells in microplates that are formed in such a manner as to facilitate storage of frozen solutions, the mixing of such solutions, after thawing, and the turbulent vortex mixing of combinations of liquids in such tubes that are to be combined for assays. FIGS. 10A and B show a microplate 90 constructed in accordance with the invention, and FIG. 10C shows detail of a cross section of two wells formed in plate 90. It is clear that this same cross-section can be formed in a tube or bottle, or strip of joined tubes and that these embodiments would lie within the scope of this invention.

According to the invention there are formed on the internal surfaces of the tubes or wells, asymmetrical protrusions 91, having a first surface 92 protruding at a relatively steep angle and a second surface 93 formed as a less steep angle. Said protrusions may be organized on the inside surface of a tube in a train with the sharp angles aligned so that the protrusions homologously follow each other, and a train of protrusions may spiral from the top, larger end of a generally conical shaped tube or well down into the bottom, along path 94 and back up towards the top along path 95 as shown. When a tube having such protrusions is subjected to vibratory motion it is clear that a liquid in the tube will be pushed down along path 94 and back up path 95. By this means simple rectilinear motion will induce vortex motion of a liquid in a tube. It is also clear that a solution that is frozen in a tube having such protrusions will be held in place in the tube and will not be able to fall out, since the said protrusions will lie within the frozen mass. A microplate constructed in accordance with the invention preferably has cuts 96 formed between rows of the plate to facilitate the breaking off of rows for their use alone, when all wells in the plate do not need to be used.

Robot arm 40 uses tips 24 to dispense and combine reagents 29, 30, 31, 32 for an assay into the wells of a storage microplate 90 in location 44 which is located on a temperature controlled microplate block 45, the temperature of such a block being controlled by the instrument microprocessor, preferably heated by resistive heating and cooled by thermoelectric cooling. After assay solutions have been made up, the individual solutions in the wells of the plate may be frozen in situ by causing the block temperature to descend below the freezing point of the mixture. This ability of the invention is beneficial and useful for preparing, freezing and storing multiple plates of assay “master-mix” before target molecules are to be extracted, or, for freezing and storing microplates that contain purified nucleic acids or other target molecules after purification, or, for freezing and storing microplates that contain complete assay mixtures of master-mix and purified nucleic acids or other target molecules. Such frozen plates may then be easily and safely transported to another location, for example, for PCR thermal cycling, without fear that small volumes of liquid will be lost en route from splashing.

I claim:

1. A process of isolating and purifying nucleic acids, proteins or other target molecules or species of molecules without the use of sedimentation, centrifugation, aspiration, air suction or vacuum, by means of the mechanical displacement of liquids and the alteration of fluid pathways within a disposable device, comprising the steps of:
(a) mixing together in an incubation vessel, in the absence of any type of nucleic acid binding solid phase, one or more lysis reagents with a sample containing nucleic acids or other target molecules;
(b) agitating, mixing and heating the lysis mixture to effect the liberation of all nucleic acids or other target molecules into solution;
(c) causing the mixture of liberated nucleic acids or other target molecules to leave the incubation vessel and to pass through a porous, solid phase filter capable of binding said liberated nucleic acids or other target molecules;
(d) washing the solid phase filter to remove impurities and enzyme inhibitors; and
(e) eluting the nucleic acids or other target molecules from the filter in a small volume.

2. An apparatus for carrying out the process according to claim 1, comprising
a) a first part which does not make physical contact with the starting sample material or molecules derived from starting materials
(b) a second disposable part which may be easily attached or detached from the first part, into which starting material, such as blood, blood plasma, urine, semen or a suspension of cells is drawn, and out of which purified nucleic acids or other target molecules are dispensed.
(c) a said first part that is able to control the internal fluid pathways of the second part by means of stepper motors that rotate stopcock handles or linear actuators that change the position of moveable valve elements in the second part
(d) a said first part employing at least one digital pump, at least one multiport valve, and at least one dual valve actuating device.
(e) a said first part having means of heating and controlling the temperature of reagents supplied to the second part, of mixing and agitating mixtures of reagents and samples in the second part

3. A machine constructed according to claim 2, for processing a starting material that is held in a capillary tube or other small tube, in which the end of the capillary tube or tip is inserted sealingly into a cooperating part of the disposable device of the invention, which device has internal fluid pathways controlled by valves, connected to at least one incubation chamber, and a fluid fitting that may be sealingly connected to the first part of the invention, which part has means for injecting gases and liquids into the device, and means for altering the fluid pathways within the device by changing the positions of valve elements within the device, together with means for heating and combining together a mixture of the starting material and a lysis reagent, according to the following steps:
(a) causing a sample to be withdrawn from a capillary tube or pipette tip into the device
(b) causing the sample to be combined with one or more lysis reagents in an incubation vessel of the device
(c) heating and agitating the lysis mixture in the incubation vessel
(d) withdrawing the lysis mixture from the incubation vessel
(e) forcing the lysis mixture to pass through a filter that is able to bind a nucleic acid or other target molecule
(f) causing one or more wash solution to enter the filter to wash the filter
(g) heating a wash solution and agitating a wash solution in the filter
(h) removing a wash solution from the filter.
(i) causing one or more elution solution to enter the filter to elute target molecules from the filter.
(j) heating an elution solution and agitating an elution solution in the filter to remove bound nucleic acids or other target molecules from the filter in a small volume

4. A machine constructed according to claim 3, in which starting material is drawn into the disposable device via a tube conduit, the end of which is placed in a liquid sample held in a sample tube or a microtiter plate.

5. A machine constructed according to claim 3, in which starting material is drawn into the disposable device via a needle which punctures the elastomeric stopper of a sample tube, including the “vacutainer” system

6. A machine constructed according to claim 3, in which a disposable device component is composed of two 4-way stopcocks and two low dead-volume tee connectors together with attached tubing, filters, and vials, the tee connectors being formed with hubs on the male luer fittings and shelves in the female luer fittings to increase the reproducibility of the internal volume of the assembly.

7. A machine constructed according to claim 3, in which the stopcock handles of a disposable device component constructed in accordance with claim 6 are rotated by two motors mounted together on a single plate with the distance between the axes of the two motors the same as the distance between the axes of the two stopcock handles; provided with drive adapters allowing each stopcock handle to snap into the adapter and thereby to be controlled by the respective motor, permitting each stopcock to be operated independently from the other and any combination of stopcock ports to be blocked or connected together.

7. A machine constructed according to claim 3, in which the disposable device component has valves that are linearly actuated and are connected by internal passages formed in a single device housing.

8. A machine for carrying out the process according to claim 1, in which one or a multitude of samples each is combined with one or more lysis reagents in one or more incubation vessels provided with means for heating and agitation of the lysis mixture; the incubation vessels may consist of rows of tubes or wells in a microtiter plate, and the means for combining the samples and lysis reagents may preferably consist of a robotic actuated pipettor using aerosol resistant tips to transfer and combine starting material and lysis reagents into the incubation vessels; the lysis solutions, after being agitated and heated, are removed from the incubation vessels by means of being drawn into aerosol-resistant tips that contain porous filters that can bind a nucleic acid or other target molecule, such filter element being located either near the end of a tip, or if at a distance, the passage connecting the filter and the end of the tip is of low volume and narrow bore and the abutting space between such passage and the filter has a low dead volume; the entire quantity of lysis solution is made to pass through the filter element on being withdrawn from an incubation vessel, and to pass again through the filter element when a solutions is dispensed out of the tip; after dispensing, the filters are washed by taking wash solutions in and out of the tips, through the filter elements and the nucleic acid or other target molecules are eluted in a small volume by picking up and causing a small volume of elution reagent to pass first into and then through each filter element, then back into and through the filter several times before dispensing nucleic acid or other target molecules in a small volume into sample vials or into mixtures of nucleic acid assay reagents.

9. An nucleic acid or other target molecule binding pipettor tip constructed in accordance with claim 5, in which a solid phase filter element capable of binding nucleic acids is located at the top of a low-internal volume extension of the entrance of the tip, or other filter elements are provided, including solid reagents above the filter element that are able to dissolve in a liquid above the filter element, thereby releasing soluble reagents or insoluble materials into such solutions.

10. An apparatus constructed according to claims 3 and 4, for processing a starting material that is held in a capillary tube, a pipette tip, a sample tube or microtiter plate, in which the disposable part of the apparatus may be provided with one or more reagents held in the incubation vessel or in one or more disposable syringes sealingly attached to said disposable part.

11. A process according to claim 1, in which the lysis reagent contains a greater than 5 molar concentration of a chaotropic reagent, such as urea, guanidine isothiocyanate or potassium iodide and a concentration of 0.1%-5% of a non-ionic detergent such as octylphenoxypolyethoxyethanol.

12. A process according to claim 1, in which the nucleic acid binding filter contains fibers or particles of polystyrene, nitrocellulose, glass, or quartz.

13. A process according to claim 1, in which target molecules are captured using lysis reagents that contain probes capable of binding to the target molecules. After agitation and heating of the lysis mixture, another reagent may be added to rapidly cool the lysis solution-target molecule mixture, and alter the solvent environment of the lysis mixture to promote binding of probes with target molecules, before causing the mixture to pass through a porous, solid phase filter capable of binding the probes and target molecules which have been captured by the said probes.

14. A process according to claim 10, in which the target molecules are nucleic acid sequences and the probes are RNA, DNA, PNA or other molecules capable of being selectively bound to target nucleic acid sequences, which probes have a second functional portion that does not selectively bind to target molecules, such as a poly A tail or a biotin molecule, and the filter element has a corresponding molecule capable of binding to the second functional portion, such as poly T nucleotides or avidin molecules which are bound to the filter. The choice of elution reagent for the target molecules depends upon the type of interaction between probe and filter that is required to be disrupted, in order to facilitate release of the target molecule. The choice of elution reagent for the target molecules depends upon the type of interaction between probe and filter that is required to be disrupted, in order to facilitate release of the target molecule.

15. A process according to claim 10, in which the target molecules are proteins, carbohydrates or other molecules and the probes are conjugate molecules, one part of a conjugate consisting of an antibody or other molecule capable of reacting with, or forming an affinity-based bond with the target molecule, the second moiety of the conjugate being capable of reversibly binding with the filter element. The choice of elution reagent for the target molecules depends upon the type of interaction between probe and filter that is required to be disrupted, in order to facilitate release of the target molecule.

16. A process according to claim 10, in which the target molecules are nucleic acid sequences, the probes are 5′-biotinylated oligonucleotides between 20 to 40 nucleotides in length that are complimentary to the target nucleic acid sequence, the second lysis reagent contains at least 0.7 molar sodium phosphate buffer, pH 7.4, and the filter for binding the probes contains modified avidin residues capable of releasing of biotin under mildly acidic conditions.

17. Separate sample tubes, or strips of connected tubes, or wells contained in a plate, such tubes or wells having formed on their internal surfaces asymmetrical protrusions, each protrusion having a first surface protruding at a relatively steep angle and a second surface protruding as a less steep angle; the said protrusions are organized on the inside surface of a tube in a train with the sharp angular faces of the protrusions aligned so that the protrusions homologously follow each other, and a train of protrusions spirals from the top, larger end of a generally conical shaped tube or well down into the bottom, then turns back up towards the top.

18. A kit for carrying out the process according to claim 1, for use with a machine constructed in accordance with claims 2, 3, 4 or 7 comprising a disposable device constructed in accordance with claims 2, 3, 4 or 7 together with such lysis, wash and elution reagents as may be required to carry out the nucleic acid purification process.

19. A kit for carrying out the process according to claim 1, for use with a machine constructed in accordance with claim 5 comprising a multiplicity of disposable devices constructed in accordance with claims 5 and 6, together with such lysis, wash and elution reagents as may be required to carry out the nucleic acid purification process.

20. An apparatus for mixing purified target molecules with components of an assay comprising
a) a first part which does not make physical contact with the starting sample material or molecules derived from starting materials
(b) a second disposable part which may be easily attached or detached from the first part, into which purified target molecules are drawn and combined with components of assays
(c) a said first part that is able to control the internal fluid pathways of the second part by means of stepper motors that rotate stopcock handles or linear actuators that change the position of moveable valve elements in the second part
(d) a said first part employing at least one digital pump, at least one multiport valve, and at least one dual valve actuating device.
(e) a said first part having means of heating and controlling the temperature of reagents supplied to the second part, of mixing and agitating mixtures of reagents and samples in the second part comprising
(f) a said first part that is able to control the operation of a testing component such as a thermal cycler, a fluorescence detector or gene chip, and by altering the internal fluid pathways of the second part cause the sample and other assay components to be processed by the said test component.

21. An apparatus constructed according to claims 2 and 20 in which the disposable second part of claim 2 is joined with the disposable second part of claim 20, thereby enabling target molecules to be purified and then subsequently to be assayed, totally under the control of the apparatus.
